(19) United States
(12) Patent Application Publication
(10) Pub. No.: US 2017/0245734 A1
(43) Pub. Date: Aug. 31, 2017

(54) RESIN DISTAL END COMPONENT FOR ENDOSCOPE

(71) Applicant: OLYMPUS CORPORATION, Tokyo (JP)

(72) Inventor: Hiroyuki KANEKO, Tokyo (JP)

(73) Assignee: OLYMPUS CORPORATION, Tokyo (JP)

(21) Appl. No.: 15/597,660

(22) Filed: May 17, 2017

Related U.S. Application Data
(63) Continuation of application No. PCT/JP2016/061029, filed on Apr. 4, 2016.

(30) Foreign Application Priority Data
May 8, 2015 (JP) 2015-095742

(51) Int. Cl. A61B 1/00; A61B 1/307; A61B 1/05; A61B 1/06; A61B 10/04; A61B 1/005; A61B 1/018

(52) U.S. Cl. CPC A61B 1/0008; A61B 1/0052; A61B 1/307; A61B 1/018; A61B 1/06; A61B 10/04; A61B 1/05; A61B 2010/045

(57) ABSTRACT

A distal end component includes: a transparent resin outer layer portion that is made of a transparent first resin member and configures a surface of a distal end portion; an internal structure portion that is made of a second resin member and is provided in tight contact with an inner surface of the transparent resin outer layer portion; a plurality of component fixing holes provided in the internal structure portion; and lens sections provided in the transparent resin outer layer portion, the lens sections respectively facing distal end surfaces of illumination optical sections and a distal end surface of an observation optical section

### CROSS REFERENCE TO RELATED APPLICATION

This application is a continuation application of PCT/JP2016/061029 filed on Apr. 4, 2016 and claims benefit of Japanese Application No. 2015-095742 filed in Japan on May 8, 2015, the entire contents of which are incorporated herein by this reference.

### BACKGROUND OF INVENTION

1. Field of the Invention

The present invention relates to a resin distal end component for an endoscope that is provided at a distal end portion of an endoscope and is formed of two kinds of resin members that are integrated.

2. Description of the Related Art

An endoscope has been widely used in a medical field and in an industrial field. A diagnosis object of the endoscope or an observation object is an inside of a living body, an inside of a plant, and the like. Therefore, a light source that applies light to the diagnosis object or the observation object is necessary for an endoscope apparatus.

A typical endoscope apparatus includes an endoscope and a light source apparatus as an endoscope external apparatus. The light source apparatus includes a light emitting source that emits illumination light, such as a lamp and a light emitting device.

The illumination light emitted from the light emitting source is transmitted through a light guide fiber provided in the endoscope, and passes through an illumination window provided at a distal end of the insertion section, thereby being applied to the observation object.

The illumination window is typically water-tightly fixed to a through hole that is provided in an insulation distal end cover. In addition, the distal end cover to which the illumination window is fixed is integrally fixed to a distal end rigid portion made of a metal such as a stainless steel. The distal end rigid portion forms the distal end portion of the insertion section.

International Publication No. 2011-111242 discloses a method of manufacturing a resin molded product and a resin molded product. The resin molded product is manufactured by integrally molding an optical device and a supporting member supporting the optical device. The optical device is used for an observation optical system of an endoscope and/or an image pickup optical system of a camera.

In FIG. 27, FIG. 35, and other drawings of International Publication No. 2011-111242, a two-color molded product in which an illumination lens is made of a resin for an optical component, and a distal end portion body serving as the supporting member is made of an optically opaque resin such as a black resin, is illustrated. The two-color molded product makes it possible to form the distal end portion of the insertion section without increasing a diameter of the insertion section of the endoscope.

### SUMMARY OF THE INVENTION

A resin distal end component for an endoscope according to an aspect of the present invention, includes: a transparent resin outer layer portion that is made of a transparent first resin member and forms a surface of a distal end portion of an insertion section; an internal structure portion that is made of a second resin member different from the first resin member, and is provided in tight contact with an inner surface of the transparent resin outer layer portion; a plurality of component fixing holes in which distal-end-side end portions of components provided in the insertion section are respectively disposed, the component fixing holes being provided in the internal structure portion; and lens sections provided in the transparent resin outer layer portion, the lens sections respectively facing distal end surfaces of optical components disposed in the insertion section, the optical components each having a distal-end-side end portion disposed in a corresponding component fixing hole of the component fixing holes.

The present invention makes it possible to realize the resin distal end component for the endoscope that is formed by integral molding of the transparent resin for the optical component and the resin different from the transparent resin and is suitable to be disposed at the distal end portion of the insertion section.

### BRIEF DESCRIPTION OF THE DRAWINGS

FIG. 1 is a diagram to explain an endoscope;

FIG. 2A is a front view of a distal end portion of an insertion section;

FIG. 2B is a diagram of the distal end portion of the insertion section including an internal structure portion, as viewed from a front side;

FIG. 3 is a cross-sectional diagram taken along arrow line Y3-Y3 in FIG. 2A;

FIG. 4 is a cross-sectional diagram taken along arrow line Y4-Y4 in FIG. 2A;

FIG. 5A is a diagram illustrating a cross-section taken along arrow line Y5-Y5 similar to the arrow line Y3-Y3 in FIG. 2A, and is a diagram to explain a distal end component that is formed of a transparent resin member and an opaque resin member;

FIG. 5B is a cross-sectional diagram taken along arrow line Y5B-Y5B in FIG. 2A, and is a diagram to explain the distal end component that is formed of the transparent resin member and the opaque resin member;

FIG. 6 is a diagram of the distal end component as viewed from a front side to explain other configuration example of the distal end component; and

FIG. 7 is a cross-sectional diagram taken along arrow line Y7-Y7 in FIG. 6.

### DETAILED DESCRIPTION OF THE PREFERRED EMBODIMENT(S)

Hereinafter, an embodiment of the present invention is described with reference to drawings.

Note that scale sizes of some components are varied in illustration for each component in order to illustrate the components with respective recognizable sizes in the drawings used in the following description. Moreover, the present invention is not limited to the number of components, shapes of the components, a size ratio of the components, and relative positional relationship between the respective components that are illustrated in the respective drawings.

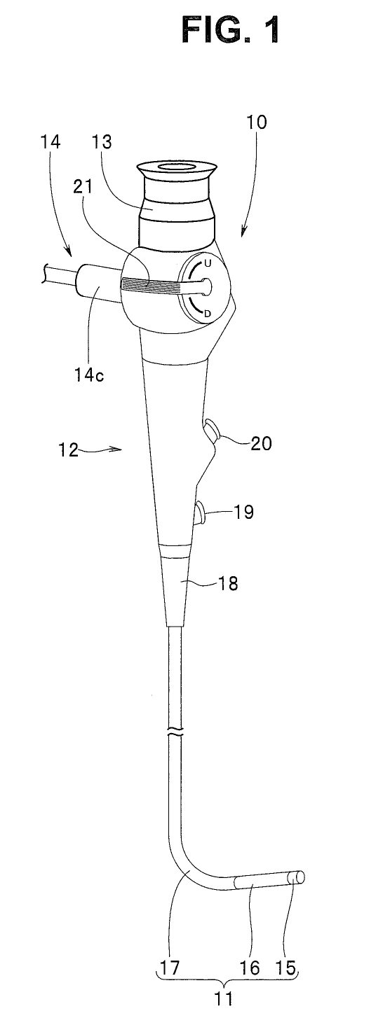

An endoscope 10 illustrated in FIG. 1 is, for example, a nephro-ureteroscope, and is mainly configured by providing an insertion section 11, an operation section 12, and an eyepiece section 13. The eyepiece section 13 is provided on proximal end side of the operation section 12.

A reference numeral 14 denotes a universal cord, and a connector 14c of the universal cord 14 is detachable to a light guide connection opening (not illustrated) that is provided on a side part of the operation section 12.

The insertion section 11 is configured by providing a rigid distal end portion 15, a bending portion 16, and a flexible tube portion 17 that are connected with one another in order from the distal end side of the insertion section 11. The bending portion 16 is so configured as to be bent in a vertical direction. The flexible tube portion 17 is a flexible tube body.

A bend preventing portion 18 having predetermined elastic force is provided on proximal end side of the flexible tube portion 17. The bend preventing portion 18 is so provided as to cover a proximal end portion of the flexible tube portion 17 to prevent buckling of the flexible tube portion 17 and to retain water-tightness between the flexible tube portion 17 and distal end side of the operation section 12.

A water leakage detection pipe sleeve 19, a treatment instrument insertion opening 20, a bending operation lever 21, and the like are provided on the operation section 12. The bending operation lever 21 is rotatable, and the bending portion 16 is bent in the vertical direction through pulling and relaxing of a bending wire (not illustrated) along with rotation operation of the lever 21.

Note that the bending portion 16 may be configured by providing an active bending portion and a passive bending portion. The active bending portion is bent through pulling and relaxing of the bending wire, and the passive bending portion is bent in response to external force. In addition, the endoscope 10 is not limited to a nephro-ureteroscope.

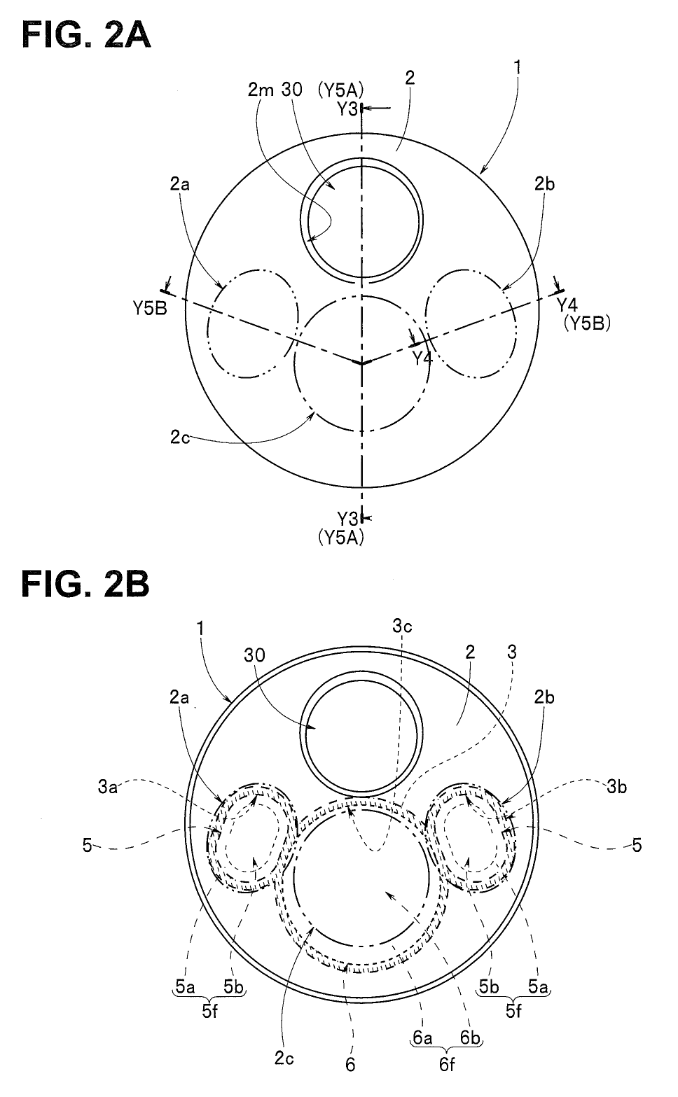

A reference numeral 2 illustrated in FIG. 2A denotes a transparent resin outer layer portion that configures a distalmost end portion of a resin distal end component for the endoscope (hereinafter, abbreviated as the distal end component). The transparent resin outer layer portion 2 is made of a first resin member, and forms a distal end surface of the distal end portion 15. The first resin member is an insulation transparent resin for an optical component.

An opening 2m of a treatment instrument channel hole 30 is provided at a predetermined position of the transparent resin outer layer portion 2 that is a front surface of the distal end component 1. In addition, illumination lens sections 2a and 2b and an observation lens section 2c that are illustrated by alternate long and two short dash lines, are provided at respective predetermined positions of the transparent resin outer layer portion 2.

In the present drawing, the two illumination lens sections 2a and 2b are so provided as to sandwich the observation lens section 2c. The number of the illumination lens sections, however, is not limited to two, and two or more or two or less illumination lens sections may be provided.

As illustrated in FIG. 2B, an internal structure portion 3 is provided on inner side of the transparent resin outer layer portion 2 in the distal end portion 15. The internal structure portion 3 includes through holes 3a, 3b, and 3c in which two illumination optical sections 5 and an observation optical section 6 are respectively disposed.

A reference numeral 5f denotes a distal end surface of each of the illumination optical sections 5, and the distal end surfaces of the illumination optical sections 5 respectively face the illumination lens sections 2a and 2b. A reference numeral 5a denotes a pipe, and a reference numeral 5b denotes a light guide fiber.

In contrast, a reference numeral 6f denotes a distal end surface of the observation optical section 6, and the distal end surface of the observation optical section 6 faces the observation lens section 2c. A reference numeral 6a denotes a first lens frame, and a reference numeral 6b denotes an observation lens.

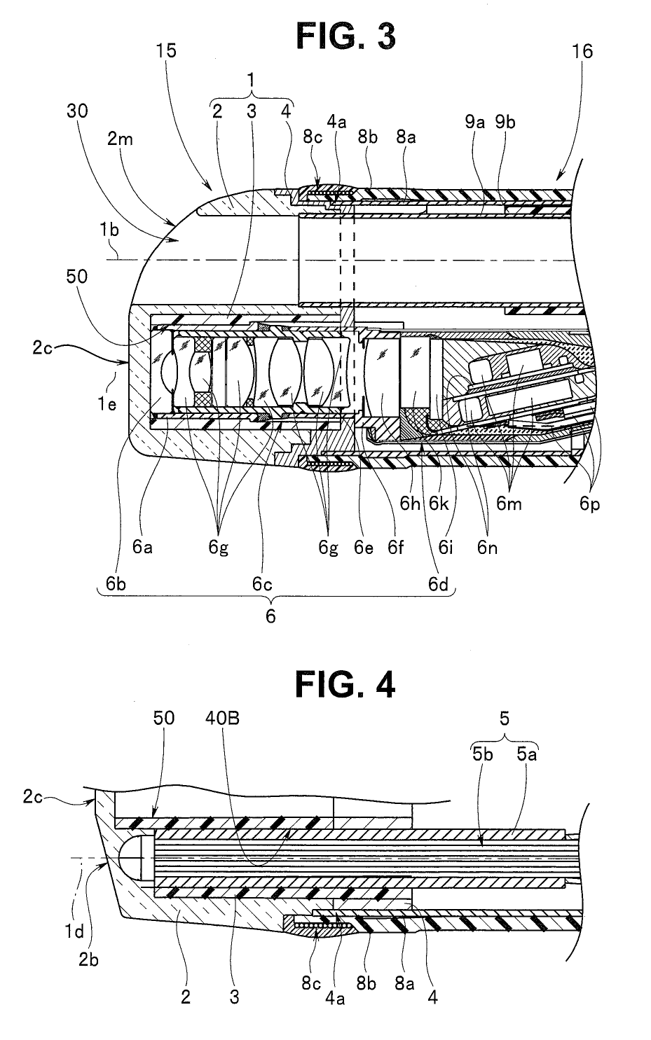

As illustrated in FIG. 3 and FIG. 4, the distal end component 1 is provided at the distal end portion 15.

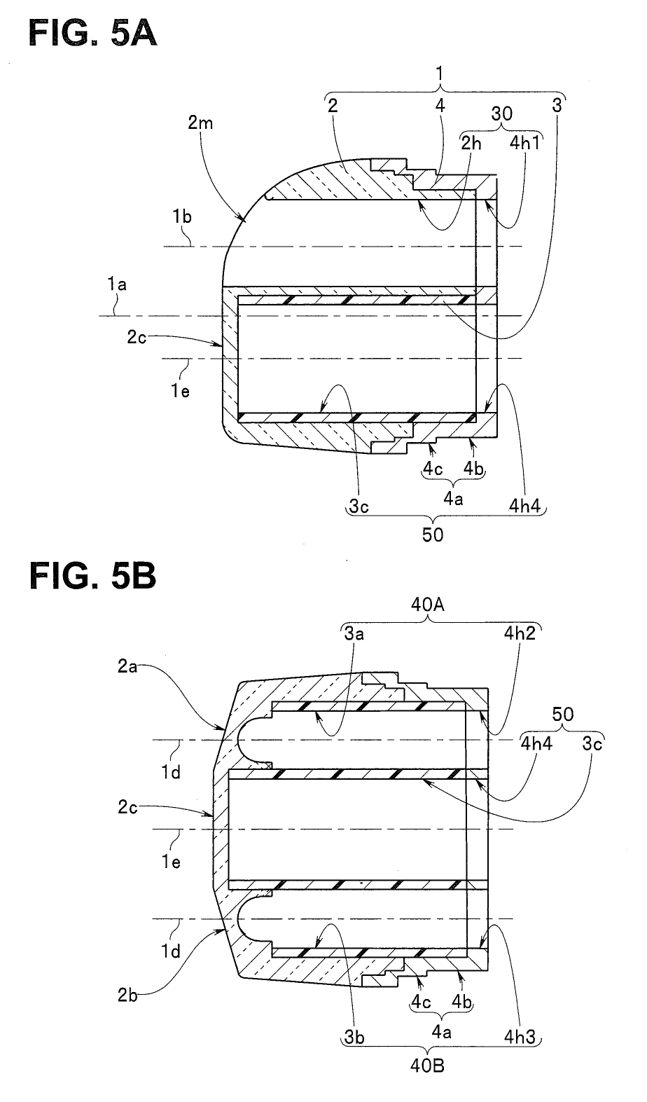

As illustrated in FIG. 5A and FIG. 5B, the distal end component 1 is a substantially cylindrical distal end constituent member that includes the transparent resin outer layer portion 2, the internal structure portion 3, and a coupling portion 4.

The internal structure portion 3 is made of a second resin member that is different from the first resin member. The internal structure portion 3 is provided in tight contact with an inner surface 2i of the transparent resin outer layer portion 2. Further, the internal structure portion 3 is so provided as not to be exposed on the distal end surface of the distal end portion 15.

The second resin member is an optically opaque insulation resin, for example, a black resin. In other words, in the present embodiment, the internal structure portion 3 functions as a light shielding section.

The transparent resin outer layer portion 2 and the internal structure portion 3 are formed as a two-color molded product 7 through integral molding.

The coupling portion 4 is a light shielding member, for example, an annular member made of a stainless steel pipe.

A peripheral step part 4a is provided on an outer peripheral surface on the proximal end side of the coupling portion 4. The peripheral step part 4a includes a first step part 4b and a second step part 4c. For example, the first step part 4b is a step part for a piece, and the second step part 4c is a step part for a rubber.

The distal end of the coupling portion 4 is disposed on an outer peripheral surface on the proximal end side of the transparent resin outer layer portion 2 that is configured as the two-color molded product 7. In the arrangement state, an inner surface of the proximal end part of the coupling portion 4 is disposed to tightly seal a boundary 7a between the internal structure portion 3 and the transparent resin outer layer portion 2 that is exposed on a proximal end surface of the two-color molded product 7.

Further, the coupling portion 4 is integrally fixed to the two-color molded product 7 by, for example, an adhesive, thereby configuring the distal end component 1. Therefore, an outer surface of the distal end component 1 is formed of the transparent resin outer layer portion 2 and the coupling portion 4.

In the distal end component 1, the treatment instrument channel hole 30 that is a through hole, and illumination optics holes 40A and 40B and an observation optics hole 50 that are blind holes are provided.

Each of the treatment instrument channel hole 30, the illumination optics holes 40A and 40B, and the observation optics hole 50 also serves as a component fixing hole.

The treatment instrument channel hole 30 has a first axis lb that is parallel to a longitudinal axis 1a of the distal end component 1. The treatment instrument channel hole 30 is formed of a through hole 2h and a treatment through hole 4h1. The through hole 2h has the distal end opening 2m provided in the transparent resin outer layer portion 2. The treatment through hole 4h1 is provided in the coupling portion 4.

The first illumination optics hole 40A is provided corresponding to the first illumination lens section 2a, and has a second axis 1c that is parallel to the longitudinal axis 1a of the distal end component 1. The first illumination optics hole 40A is formed of a first through hole 3a of the internal structure portion 3 and a first illumination optics through hole 4h2. The first illumination optics through hole 4h2 is provided in the coupling portion 4.

The second illumination optics hole 40B is provided corresponding to the second illumination lens section 2b, and has a third axis 1d that is parallel to the longitudinal axis 1a of the distal end component 1. The second illumination optics hole 40B is formed of a second through hole 3b of the internal structure portion 3 and a second illumination optics through hole 4h3. The second illumination optics through hole 4h3 is provided in the coupling portion 4.

The observation optics hole 50 is provided corresponding to the observation lens section 2c, and has a fourth axis le that is parallel to the longitudinal axis la of the distal end component 1. The observation optics hole 50 is formed of a third through hole 3c of the internal structure portion 3 and an observation optics through hole 4h3. The observation optics through hole 4h3 is provided in the coupling portion 4.

As illustrated in FIG. 3 and FIG. 4, a distal end bending piece 8a of an unillustrated bending portion set forming the bending portion 6 is disposed at the step part for the piece of the peripheral step part 4a, and a bending rubber 8b is disposed at the step part for the rubber. The bending rubber 8b covers the bending portion set to form the bending portion 16.

A reference numeral 8c denotes a bobbin adhesion fixing portion.

As illustrated in FIG. 4, the distal end portions of the illumination optical sections 5 that are optical components are respectively fixed to the illumination optics holes 40A and 40B.

Each of the illumination optical sections 5 includes a pipe 5a and a light guide fiber 5b. A distal end portion of the light guide fiber 5b is protected by the pipe 5a.

In contrast, as illustrated in FIG. 3, a distal end portion of a tube pipe sleeve 9a that is one of components is fixed to the proximal end side in the treatment instrument channel hole 30. A distal end portion of a channel tube 9b is externally fitted to a proximal end portion of the tube pipe sleeve 9a.

A treatment instrument such as a biopsy needle is guided into the channel tube 9b from the treatment instrument insertion opening 20 illustrated in FIG. 1, is guided into the treatment instrument channel hole 30 through the tube pipe sleeve 9a, and is then guided out from the distal end opening 2m.

A distal end portion of the observation optical section 6 that is one optical component is fixed to the observation optics hole 50.

The observation optical section 6 is mainly configured by having an observation lens 6b, an objective optical system 6c, an image pickup unit 6d, and the like. A reference numeral 6a denotes a first lens frame, a reference numeral 6e denotes a second lens frame, and a reference numeral 6f denotes an image pickup frame.

The observation lens 6b is disposed inside the first lens frame 6a. A plurality of kinds of optical lenses 6g, a spacer ring (not illustrated), a diaphragm (not illustrated), and the like are disposed as optical members inside the second lens frame 6e. A distal end portion of the second lens frame 6e is fixed to the first lens frame 6a. An optical lens 6h that is an optical member, and the like are disposed inside the image pickup frame 6f. A proximal end portion of the second lens frame 6e is fixed to the image pickup frame 6f.

The image pickup unit 6d includes: a solid-state image pickup device 6i such as a CCD and a CMOS; a cover glass 6k that is disposed in front of the image pickup surface; a circuit substrate 6n mounted with a plurality of electronic parts 6m; a signal cable (not illustrated) that is formed of a bundle of a plurality of signal lines 6p each connected to the circuit substrate 6n; and other components.

According to the endoscope 10 having the above-described configuration, when illumination light is emitted from an unillustrated light source apparatus, the illumination light is transmitted by the light guide fiber 5b that is provided in the endoscope 10. The illumination light is then outputted from the illumination optical sections 5 that are respectively disposed in the illumination optics holes 40A and 40B of the distal end component 1 configuring the distal end portion 15 of the insertion section 11.

The illumination light outputted from the distal ends of the respective illumination optical sections 5 is diffused by concave parts provided on lens rear surfaces of the respective illumination lens sections 2a and 2b. The diffused light is applied from lens front surfaces of the respective illumination lens sections 2a and 2b to the inside of a body cavity after passing through the transparent resin outer layer portion 2, thereby illuminating an observation site with appropriate light distribution.

As a result, an optical image of the observation site illuminated with the illumination light passes from the lens front surface of the observation lens section 2c through the transparent resin outer layer portion 2, then passes through the lens rear surface to enter the observation optical section 6, thereby being picked up by the image pickup unit 6d.

As a result, intended optical performance is realized, and an excellent endoscope image is displayed on a screen of an unillustrated display apparatus.

The endoscope 10 after use is sterilized in an autoclave.

In the present invention, the distal end component 1 in which the coupling portion 4 is integrally fixed to the two-color molded product 7 is provided at the distal end portion 15 of the insertion section 11. The two-color molded product 7 is formed of the transparent resin outer layer portion 2 and the internal structure portion 3 through two-color molding. In addition, the outer surface of the distal end component 1 is formed of the coupling portion 4 and the transparent resin outer layer portion 2 that has no boundary between the first resin member and the second resin member because of the two-color molding.

Such a configuration makes it possible to dispose only the transparent resin outer layer portion 2 of the two-color molded product 7 on the outer surface of the distal end component 1. This eliminates crack that occurs from a notch as a starting point when the autoclave sterilization is repeatedly performed. The notch is a fine groove.

In addition, configuring the internal structure portion 3 as the light shielding section makes it possible to surely prevent the illumination light of the illumination optical sections 5 provided in the respective through holes 3a and 3b of the internal structure portion 3, from being outputted to the outside through the internal structure portion 3.

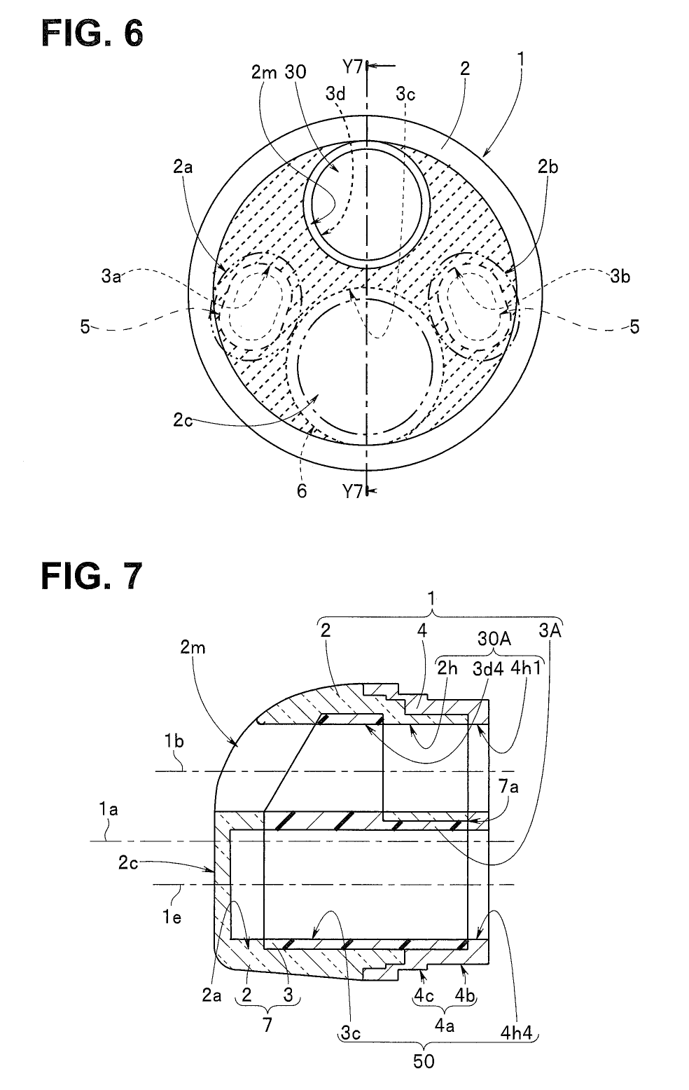

Note that, in the above-described embodiment, the internal structure portion 3 includes the through holes 3a, 3b, and 3c as illustrated in FIG. 2B. As illustrated in FIG. 6 and FIG. 7, however, an internal structure portion 3A that includes a fourth through hole 3d in addition to the through holes 3a, 3b, and 3c may be adopted.

The fourth through hole 3d is a treatment instrument insertion through hole.

An outer shape of the internal structure portion 3 is not limited to the shape in the above-described embodiment. Other configurations of the internal structure portion 3 are similar to the configurations in the above-described embodiment, and the same components are denoted by the same reference numerals as the above-described embodiment and description of such components is omitted.

In the present embodiment, a treatment instrument channel hole 30A is composed of: the through hole 2h that has the distal end opening 2m provided in the transparent resin outer layer portion 2; the fourth through hole 3d of the internal structure portion 3A; and the treatment through hole 4h1 provided in the coupling portion 4.

The configuration makes it possible to provide action and effects similar to the action and effects of the above-described embodiment.

Note that the present invention is not limited only to the above-described embodiments, and may be variously modified without departing from the scope of the invention.

What is claimed is:

1. A resin distal end component for an endoscope, comprising:
a transparent resin outer layer portion that is made of a transparent first resin member and forms a surface of a distal end portion of an insertion section;
an internal structure portion that is made of a second resin member different from the first resin member, and is provided in tight contact with an inner surface of the transparent resin outer layer portion;
a plurality of component fixing holes in which distal-end-side end portions of components provided in the insertion section are respectively disposed, the component fixing holes being provided in the internal structure portion; and
lens sections provided in the transparent resin outer layer portion, the lens sections respectively facing distal end surfaces of optical components disposed in the insertion section, the optical components each having a distal-end-side end portion disposed in a corresponding component fixing hole of the component fixing holes.

2. The resin distal end component for the endoscope according to claim 1, wherein one or more of the component fixing holes are optical component fixing holes in which the optical components are respectively disposed, each of the optical components including an optical section that faces a corresponding lens section of the lens sections provided in the transparent resin outer layer portion.

3. The resin distal end component for the endoscope according to claim 1, further comprising:
a two-color molded product that is formed of the transparent resin outer layer portion and the internal structure portion serving as a light shielding section; and
a coupling portion having a distal end portion, the distal end portion being disposed on an outer peripheral surface on proximal end side of the transparent resin outer layer portion of the two-color molded product.

4. The resin distal end component for the endoscope according to claim 3, wherein the coupling portion serves as a light shielding member.
